(19) United States
(12) Patent Application Publication
(10) Pub. No.: US 2006/0142394 A1
(43) Pub. Date: Jun. 29, 2006

(54) Use of proteases for decomposing allergens

(76) Inventors: Carl-Heinz Kapitz, Kronau (DE); Gerhard Knapp, Viernheim (DE)

Correspondence Address: WILLIAM COLLARD;COLLARD & ROE, P.C., 1077 NORTHERN BOULEVARD, ROSLYN, NY, 11576, US

(21) Appl. No.: 10/526,054

(22) PCT Filed: Aug. 30, 2002
(86) PCT No.: PCT/EP02/09684

§ 371 Date: Oct. 7, 2005

(30) Foreign Application Priority Data
Aug. 28, 2002 (WO) PCT/EP02/09598

(51) Int. Cl. A61K 31/16

(52) U.S. Cl. 514/625

(57) ABSTRACT

The invention relates to the use of an active ingredient for inhibiting dust mite feces and/or mold spores as well as for denaturing the keratin of animal hair as well as plant pollen or plant spores, consisting of an enzyme such as auxillase, alcalase, bromelain, alpha-chymotrypsin, collagenase, pepsin, pronase, thrombin and trypsin, especially the enzyme auxillase, preferably consisting also of water, anionic surfactants, fragrances and preservatives, packaged in containers used for spraying or squirting, or placed into textiles, textile materials or bags, in liquid or paste or gel form.

### TECHNICAL AREA

The invention relates to the use of an active ingredient for inhibiting dust mite feces and/or mold spores and/or for denaturing the keratin of animal hair as well as plant pollen or plant spores, according to the generic part of claim 1 as well as of claim 2.

### STATE OF THE ART

All kinds of allergies are on the rise, whereby dust mites are one of the causes of these allergies. Dust mites can be found everywhere. Dust mites feed on human epidermal scales, which consist mainly of keratin protein. Dust mites as such do not cause allergies. What triggers allergies in humans is the feces excreted by dust mites, which consists mainly of proteins (proteids), that is to say, primarily of polypeptides in the form of protein chains. By the same token, spores of molds and the keratin found in the finest of animal hair as well as numerous types of plant pollen or plant spores can trigger allergies.

Up until now, anti-dust mite agents that only kill off the dust mites are used to combat allergies of this type. In this context, DE 4202549 A1, for example, discloses a method and an agent to eliminate household dust mites and their residues found in housing units, while DE 3430611 A1 discloses an agent to kill household dust mites as well as the use of such an agent. By the same token, U.S. Pat. No. 6,130,253 discloses terpenes in aqueous solutions for purposes of killing dust mites, among others.

U.S. Pat. No. 5,679,630 discloses a cleaning composition containing, among other active ingredients, between 0.0001% to 10% of protease enzyme, which is an N76D/S103A/V104I subtilisin variant derived from Bacillus lentus subtilisin.

Moreover, EP 0425018 B1 describes a cleaning composition as a laundry detergent comprising a first endoglycosidase enzyme of Type II and, as a second enzyme, subtilisin or mutated subtilisin from the group of proteases, lipases, nucleases, glycosidases, which are different from the first enzyme; preferably an exoglycosidase and combinations thereof are selected.

### TECHNICAL OBJECTIVE

The objective of the invention is to use an agent or an active ingredient combination that renders harmless dust mite feces or mold spores as well as fine animal hairs or plant pollen, all of which are allergenic in humans.

### DISCLOSURE OF THE INVENTION AND OF ITS ADVANTAGES

This objective is achieved by using an active ingredient for inhibiting dust mite feces and/or mold spores, whereby an enzyme is used as the active ingredient that is capable of breaking down the polypeptides of the dust mite feces—which are in the form of protein chains—into oligopeptides, monopeptides, dipeptides or tripeptides or of likewise breaking down the mold spores, said breakdown products no longer having an allergenic effect on humans.

The objective is also achieved through the use of an active ingredient for denaturing the keratin of animal hair and/or for denaturing plant pollen or plant spores, whereby an enzyme is used as the active ingredient that is capable of breaking down the protein chains of the keratin of animal hair or the protein chains of plant pollen or plant spores or else the plant pollen or plant spores into oligopeptides, monopeptides, dipeptides or tripeptides, said breakdown products no longer having an allergenic effect on humans.

In another advantageous embodiment of the invention, one of the enzymes auxillase, alcalase, bromelain, alpha-chymotrypsin, collagenase, pepsin, pronase, thrombin, pancreatin or trypsin is used as the active ingredient.

In another advantageous embodiment of the invention, the active ingredient consists of an active ingredient combination of one of the employed enzymes auxillase, alcalase, bromelain, alpha-chymotrypsin, collagenase, pepsin, pronase, thrombin, pancreatin or trypsin and water.

In another advantageous embodiment of the invention, anionic surfactants are additionally added to the active ingredient combination of one of the enzymes auxillase, alcalase, bromelain, alpha-chymotrypsin, collagenase, pepsin, pronase, pancreatin, thrombin or trypsin and water.

In another advantageous embodiment of the invention, fragrances and/or preservatives are additionally added to the above-mentioned active ingredient combinations of one of the enzymes auxillase, alcalase, bromelain, alpha-chymotrypsin, collagenase, pepsin, pronase, pancreatin, thrombin or trypsin and/or water and/or anionic surfactants.

In another advantageous embodiment of the invention, the enzymes auxillase, alcalase, bromelain, alpha-chymotrypsin, collagenase, pepsin, pronase, thrombin, trypsin can be used in mixtures of two or more of the above-mentioned enzymes.

In a highly advantageous and preferred use of an active ingredient for inhibiting dust mite feces and/or mold spores and the keratin of animal hair and/or plant pollen, the enzyme auxillase is used as the active ingredient, which breaks down or at least partially denatures the polypeptides of the dust mite feces, of the mold spores and of the keratin of animal hair and/or plant pollen or plant spores. Advantageously, the active ingredient consists of an active ingredient combination of the enzyme auxillase and water, such as laboratory water (bidistilled water).

In particular through the enzyme auxillase, the polypeptides of the dust mite feces in the form of protein chains are broken down or denatured into oligopeptides, such as monopeptides, dipeptides or tripeptides, and so is the keratin of animal hair and/or plant spores, said breakdown products no longer having an allergenic effect on humans. However, it is also possible to use other proteases or enzymes that are capable of breaking down the specific polypeptides of the dust mite feces—which are in the form of protein chains—into oligopeptides, monopeptides, dipeptides or tripeptides as well as the mold spores and the keratin of animal hair and/or plant pollen. Endopeptidases as well as exopeptidases can be used as proteases.

Preferably, the enzyme auxillase or one of the enzymes employed in the active ingredient combination is present in a content ranging from 0.1% to 10%.

Moreover, in another embodiment of the invention, the total content of water in the active ingredient combination ranges from 1% to 85%. By the same token, the total content of anionic surfactants in the active ingredient combination preferably amounts to between 3% and 15%. In another embodiment of the invention, the total content of fragrances in the active ingredient combination preferably amounts to between 1% and 3%. In another embodiment of the invention, the total content of preservatives in the active ingredient combination preferably amounts to between 0.5% and 3%.

The above-mentioned percentages of the amounts contained in the active ingredient combination can also be used for the likewise mentioned enzymes alcalase, bromelain, alpha-chymotrypsin, collagenase, pepsin, pronase, thrombin or trypsin. Thus, for example, one of the enzymes auxillase, alcalase, bromelain, alpha-chymotrypsin, collagenase, pepsin, pronase, thrombin, trypsin, but preferably the enzyme auxillase, is present in the active ingredient combination in an amount of 0.1% to 10%, or the mixture of two or more of the above-mentioned enzymes is present in the active ingredient combination in an amount of 0.1% to 10%.

All of the figures indicating substance amounts in the active ingredient combination are given in percent by weight.

In another embodiment of the invention, the active ingredient combination is filled into containers used for spraying or squirting. Moreover, the container used for spraying can be a nasal spray by means of which the active ingredient combination can be sprayed directly onto the nasal mucous membranes. Or else the active ingredient combination is placed into containers such as bags, or in textiles or textile materials.

The active ingredient combination can be present in liquid or paste or gel form. For example, a spray or pressure container can contain the active ingredient combination in liquid or paste or gel form so that the active ingredient combination can be applied directly to the nasal mucous membranes by spraying or by distributing the paste or gel.

In particular, the enzyme auxillase can be used as a specific substance in the active ingredient combination for inhibiting dust mite feces and/or mold spores, whereby an example of a formulation in percent by weight is given below for the protease auxillase:

| | |
|---|---|
| water | 1.0% to 85% |
| auxillase | 0.1% to 10% |
| anionic surfactant | 3.0% to 15% |
| fragrances | 0.1% to 3% |
| preservatives | 0.5% to 3% |

This aqueous active ingredient combination can be filled into containers used for spraying or squirting. Other forms of application are also possible, namely, for example, the active ingredient combination can be filled into or applied onto bags, textiles or textile materials.

Dust mite feces can be found especially in mattresses or beds. After these surfaces have been sprayed with the active ingredient combination, the dust mite feces are simply vacuumed up after a certain exposure time. This procedure can be repeated as often as desired without any concerns since the agent is purely biological and harmless for humans.

### INDUSTRIAL APPLICABILITY

The subject matter of the invention can be used industrially for inhibiting dust mite feces and/or mold spores as well as for denaturing the keratin of animal hair and plant pollen or plant spores and thus makes a contribution to minimizing allergies to dust mite feces, molds, extremely fine animal hair and plant pollen or plant spores. The special advantage of the invention lies in the fact that the enzymatic agent employed breaks down the dust mite feces or mold spores or keratin or plant pollen or plant spores, which consist primarily of polypeptides, said breakdown products no longer having an allergenic effect on humans.

1-17. (canceled)

18. A method for inhibiting dust mite feces or mold spores, comprising applying to an affected area an active ingredient combination comprising at least one enzyme capable of breaking down polypeptides in the dust mite feces into oligopeptides, monopeptides, dipeptides or tripeptides or of breaking down the mold spores, to eliminate allergenic effects on humans, wherein the enzymes are selected from the group consisting of auxillase, alcalase, bromelain, alphachymotrypsin, collagenase, pepsin, pronase, pancreatin, thrombin and trypsin.

19. A method for denaturing the keratin of animal hair or for denaturing plant pollen or plant spores, comprising applying to an affected area an active ingredient combination comprising at least one enzyme capable of breaking down protein chains of the keratin of animal hair or protein chains of plant pollen or plant spores into oligopeptides, monopeptides, dipeptides or tripeptides, to eliminate allergenic effect on humans, said enzymes being selected from the group consisting of auxillase, alcalase, bromelain, alphachymotrypsin, collagenase, pepsin, pronase, pancreatin, thrombin and trypsin.

20. The method according to claim 18, wherein the active ingredient combination further comprises water.

21. The method according to claim 19, wherein the active ingredient combination further comprises water.

22. The method according to claim 20, wherein the active ingredient combination further comprises anionic surfactants.

23. The method according to claim 21, wherein the active ingredient combination further comprises anionic surfactants.

24. The method according to claim 18, wherein the active ingredient combination further comprises fragrances or preservatives.

25. The method according to claim 19, wherein the active ingredient combination further comprises fragrances or preservatives.

26. The method according to claim 18, wherein the active ingredient combination comprises two or more of said enzymes.

27. The method according to claim 19, wherein the active ingredient combination comprises two or more of said enzymes.

28. The method according to claim 18, wherein one of said at least one enzyme is present in the active ingredient combination in an amount of 0.1% to 10% by weight.

29. The method according to claim 19, wherein one of said at least one enzyme is present in the active ingredient combination in an amount of 0.1% to 10% by weight.

30. The method according to claim 26, wherein said two or more enzymes are present in the active ingredient combination in an amount of 1 to 10% by weight.

31. The method according to claim 27, wherein said two or more enzymes are present in the active ingredient combination in an amount of 1 to 10% by weight.

32. The method according to claim 20, wherein the water is present in amounts between 1% and 85% by weight.

33. The method according to claim 21, wherein the water is present in amounts between 1% and 85% by weight.

34. The method according to claim 22, wherein the anionic surfactants are present in the active ingredient combination in amounts between 3% and 15% by weight.

35. The method according to claim 23, wherein the anionic surfactants are present in the active ingredient combination in amounts between 3% and 15% by weight.

36. The method according to claim 24, wherein the fragrances are present in the active ingredient combination in amounts between 1% and 3% by weight.

37. The method according to claim 25, wherein the fragrances are present in the active ingredient combination in amounts between 1% and 3% by weight.

38. The method according to claim 24, wherein the preservatives are present in the active ingredient combination in amounts between 0.5% and 3% by weight.

39. The method according to claim 25, wherein the preservatives are present in the active ingredient combination in amounts between 0.5% and 3% by weight.

40. The method according to claim 18, further comprising the step of filling the active ingredient combination into containers used for spraying or squirting.

41. The method according to claim 19, further comprising the step of filling the active ingredient combination into containers used for spraying or squirting.

42. The method according to claim 18, further comprising the step of placing the active ingredient combination into containers, textiles or textile materials.

43. The method according to claim 19, further comprising the step of placing the active ingredient combination into containers, textiles or textile materials.

44. The method according to claim 41, wherein the container used for spraying is a nasal spray by means of which the active ingredient combination is sprayed directly onto nasal mucous membranes.

45. The method according to claim 42, wherein the container used for spraying is a nasal spray by means of which the active ingredient combination is sprayed directly onto nasal mucous membranes.

46. The method according to claim 18, wherein the active ingredient combination is present in liquid or paste or gel form.

47. The method according to claim 19, wherein the active ingredient combination is present in liquid or paste or gel form.
